(19) United States
(12) Patent Application Publication
(10) Pub. No.: US 2026/0279512 A1
(43) Pub. Date: Sep. 17, 2026

(54) System and Method for Displaying Computational Chemistry Information using a Data Pipeline and AI Language Models

(71) Applicants: Javier Tordable, Boston, MA (US); Pablo Villanueva, Boston, MA (US); Revaz Lobzhanidze, Boston, MA (US)

(72) Inventors: JAVIER TORDABLE, SEATTLE, WA (US); PABLO VILLANUEVA, SAN PEDRO DE LATARCE (ES); REVAZ LOBZHANIDZE, TBILISI (GE)

(73) Assignee: Pauling Inc., Boston, MA (US)

(21) Appl. No.: 19/558,092

(22) Filed: Mar. 5, 2026

Related U.S. Application Data
(60) Provisional application No. 63/769,957, filed on Mar. 11, 2025.

(51) Int. Cl. G16C 20/80; G06F 30/27; G16C 20/70

(52) U.S. Cl. CPC G16C 20/80; G06F 30/27; G16C 20/70

(57) ABSTRACT

Systems and methods are disclosed for presenting computational chemistry and molecular modeling information within an interactive conversational interface. A backend data pipeline generates and stores computational artifacts associated with molecules and/or molecule-receptor complexes, including structural representations and visualization assets. A conversational agent comprising an AI language model generates a natural-language response that includes one or more machine-interpretable visualization directives embedded in the response, each visualization directive comprising a directive type and an artifact reference corresponding to a respective artifact. A client application parses the response, identifies the visualization directives, resolves the artifact references to obtain renderable representations or resource locators, and renders corresponding 2D and/or 3D visualizations inline within the conversational interface, including within tabular content. In some embodiments, resolution is performed by a resolver service that returns a renderable resource locator, such as a signed URL or a streaming endpoint, and rendering metadata and enforces authorization and artifact-type restrictions. The disclosed approach improves reliability, scalability, and usability of conversational scientific interfaces by deterministically binding narrative text to specific computational artifacts.

### 1. FIELD OF THE INVENTION

The present invention relates to user interfaces for scientific and technical computing applications, and more particularly to systems and methods for presenting computational chemistry and molecular modeling information, including molecular visualizations and analysis artifacts, within an interactive conversational interface. The invention further relates to integrating data pipelines with AI-assisted agents and language models to generate responses that include machine-interpretable visualization directives enabling a client application to retrieve and render scientific artifacts.

### 2. BACKGROUND OF THE INVENTION

Computational chemistry and molecular simulations are powerful tools for drug discovery and development. These simulations generate large amounts of data and artifacts, including molecular structures, binding energies or scores, tabular summaries, and visualizations of ligand-protein interactions, often in multiple file formats. However, displaying and interpreting this information effectively can be challenging. Existing solutions often involve complex and cumbersome interfaces that require specialized expertise and repeated context switching between analysis tools, file viewers, and reporting workflows. Traditional methods can involve tedious manual processes, software setup and compatibility requirements, and scalability limitations when reviewing many candidate ligands or simulation outputs.

Conversational interfaces can make scientific results more accessible, but free-form natural-language responses do not reliably bind narrative explanations to specific visual artifacts, and can require manual steps to locate, open, and render the correct structures or views. There is a need for a more intuitive and accessible way to present docking and related computational chemistry information to users, including non-experts, while enabling reliable retrieval and rendering of the underlying scientific artifacts within an interactive conversational interface.

### DEFINITIONS

Artifact Reference. As used herein, an “artifact reference” (also referred to as an “artifact identifier”) comprises a machine-readable reference to an analysis or visualization artifact, including, without limitation, a filesystem path, a URL or object-storage URI, a database key, or an opaque handle or token resolvable by a server or resolver service to locate and retrieve the artifact.

Visualization Directive. As used herein, a “visualization directive” comprises a machine-interpretable token, tag, or markup element embedded in a natural-language response that specifies (i) a directive type corresponding to a visualization or rendering operation and (ii) an associated artifact reference to be retrieved and rendered by a client application.

### 3. SUMMARY OF THE INVENTION

The present invention provides systems and methods for presenting computational chemistry and molecular modeling outputs within an interactive conversational user interface. In various embodiments, a backend data pipeline generates, stores, and indexes computational artifacts associated with one or more molecules and/or molecule-receptor complexes, including structural representations and visualization assets. A conversational agent comprising an AI language model receives a user query and produces a natural-language response that includes one or more machine-interpretable visualization directives referencing the indexed artifacts. The visualization directives implement a lightweight, standardized binding between narrative text and specific scientific artifacts, enabling a client application to reliably identify what should be displayed and how it should be displayed.

In some embodiments, each visualization directive includes at least (i) a directive type corresponding to a visualization operation and (ii) an artifact reference identifying a renderable artifact. The client application parses the visualization directives, validates the directive type and artifact reference, resolves the artifact reference via a resolver service or lookup mechanism, and renders the corresponding visualization inline within the conversational interface. The artifact reference may comprise, without limitation, a filesystem path, URL, object-storage URI, database key, or an opaque handle or token resolved server-side. Multiple directives may be included in a single response to present sets of results such as ranked ligand lists, alternative poses, or related analyses, without requiring manual context switching between external visualization tools.

By providing a deterministic directive-and-resolution mechanism that integrates data pipelines, AI-assisted response generation, and inline rendering, the invention improves the usability, scalability, and reliability of conversational scientific interfaces, including for users who are not experts in computational chemistry.

### 4. DETAILED DESCRIPTION OF THE INVENTION

### System Overview

The system operates through an automated workflow consisting of the following components:
4.1 Data Pipeline: This component collects, processes, and stores chemical data. Crucially, the processed data is stored as artifacts, and one or more artifact references (e.g., file paths, URIs, database keys, or opaque handles) corresponding to such artifacts are made available to the agent and/or language model for inclusion in visualization directives. The pipeline may include data ingestion, transformation and storage, and also generation of novel data. This data is stored in a variety of formats, such as JSON and XML.
4.2 AI Language Model: This component acts as an interface for users. It is trained to understand natural language queries.
   Interpret Queries: The Language Model processes user input and determines the right course of action, in particular displaying chemical data.
   Directive Generation: This is the key innovation. Based on the user query and the available artifacts and associated metadata, the AI generates visualization directives for rendering in a specific format. For 2D visualization, it generates a visualization directive such as ![LIGAND_IMAGE](artifact_reference), where artifact_reference identifies the 2D structure artifact (e.g., encoded structure or resolver-addressable artifact). For 3D visualization, it generates a visualization directive such as ![LIGAND_VISUALIZATION](artifact_reference), where artifact_reference identifies the 3D visualization artifact (e.g., resolver-addressable asset and/or viewer state). These directives are designed to be parseable by the visualization module for deterministic retrieval and rendering.
   Response Generation: The Language Model will take all the components generated, and will return a complete response to the user.
   In various embodiments, (i) the language model inserts visualization directives directly, (ii) the language model outputs structured intent (e.g., JSON) and the agent inserts visualization directives, or (iii) the agent generates visualization directives via tool calls and the language model generates narrative text associated with the directives.
4.3 Visualization Module: This component renders 2D and 3D visualizations based on the visualization directives generated by the AI language model. It parses the visualization directives and renders corresponding artifacts.
   2D Rendering: The ![LIGAND_IMAGE](artifact_reference) directive includes an artifact reference (e.g., path, URI, database key, or opaque handle) used to retrieve and render the artifact and displays the 2D molecular structure. In particular these are graph representations of molecule topology.
   3D Rendering: The ![LIGAND_VISUALIZATION](artifact_reference) directive includes an artifact reference (e.g., path, URI, database key, or opaque handle) used to retrieve and render the artifact and display the interactive 3D molecular model. The 3D rendering may include, without limitation, stick, ball-and-stick, ribbon, surface (e.g., solvent-accessible or electrostatic), and other molecular representations.
   These visualizations are displayed in the context of a Graphical User Interface, which is web-based and displays other related content to the user.
4.4 Resolver service. In some embodiments, the system includes a resolver service configured to receive an artifact reference (or components thereof, such as ligand_uuid and context_uuid) and return a renderable resource locator (e.g., a signed URL or streaming endpoint) and optional rendering metadata (e.g., artifact_type, format, viewer state). The resolver service may enforce authorization, tenancy boundaries, allowlisted artifact types, and size limits prior to returning any locator.

### 5. DETAILED DESCRIPTION OF THE DRAWINGS

FIG. 1: A block diagram of the system architecture according to an embodiment of the present invention.

FIG. 2: A flowchart illustrating the method for displaying chemistry information within a conversational interface.

FIG. 3: An example user interface showing the display of a ligand visualization within a chat message.

FIG. 4: A detailed representation of the data flow from the backend pipeline to the frontend display.

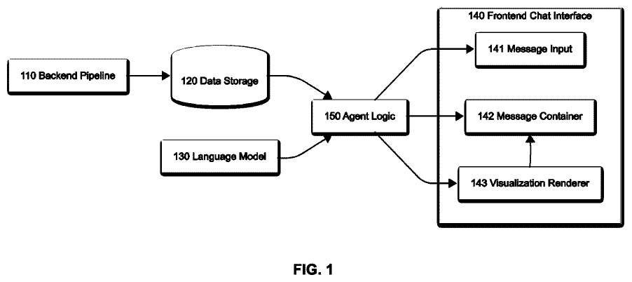

### 5.1 SYSTEM ARCHITECTURE (FIG. 1): SYSTEM ARCHITECTURE FOR DISPLAYING CHEMICAL INFORMATION WITHIN A CONVERSATIONAL INTERFACE USING AI LANGUAGE MODELS

FIG. 1 illustrates the system architecture 100 for displaying chemistry information. The system includes the following components:
Backend Pipeline 110: A computational chemistry pipeline that generates chemical data, including ligand-protein complexes, binding energies, and 2D ligand visualizations (e.g., SVG files). This pipeline may be implemented using tools such as AutoDock Vina, Open Babel, and RDKit.
Data Storage 120: A database or file system for storing the chemistry data and visualizations generated by the backend pipeline.
Language Model Integration 130: A language model capable of generating text responses based on user input. The model is configured to recognize special tags (![LIGAND_IMAGE](artifact_reference) and ![LIGAND_VISUALIZATION](artifact_reference)) that indicate the insertion points for visualizations.
Frontend Chat Interface 140: A JavaScript-based chat interface that allows users to interact with the system and view the docking information. This frontend includes:
   Message Input 141: A text input field for users to enter their queries.
   Message Container 142: A display area for showing chat messages, including text and visualizations.
   Visualization Renderer 143: A JavaScript component that recognizes the special visualization tags and renders the corresponding visualizations.
AI Agent Logic 150: The AI Agent processes user messages and orchestrates the interaction between the language model and the user of the system.

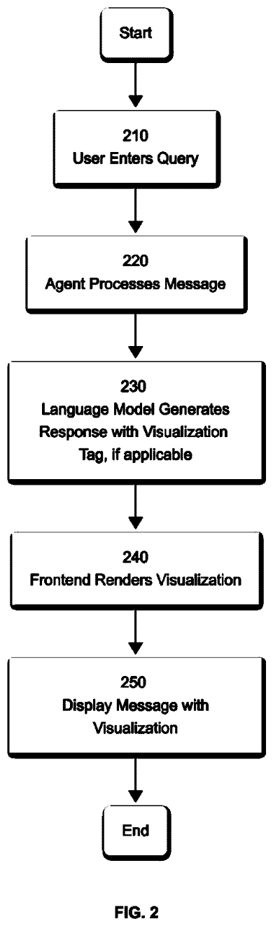

### 5.2 METHOD OF OPERATION (FIG. 2): METHOD FOR DISPLAYING CHEMICAL INFORMATION WITHIN A CONVERSATIONAL INTERFACE USING AI LANGUAGE MODELS

FIG. 2 illustrates the method 200 for displaying chemical information within the conversational interface:
Step 210: User Input: The user enters a query through the frontend chat interface (140).
Step 220: Message Processing: The AI Agent (150) receives and processes the user's message.
Step 230: Language Model Interaction: The agent interacts with the language model (130) to generate a text response. The Language Model is instructed to insert the tag as appropriate by the Agent.
Step 240: Visualization Rendering: The frontend JavaScript code (140) receives the text response and parses it. The custom image renderer recognizes the LIGAND_IMAGE tag and the LIGAND_VISUALIZATION tag. Then it displays the right information from the referenced artifact using the appropriate visualization libraries.
Step 250: Display to User: The visualization and associated text are displayed to the user within the chat interface (140).

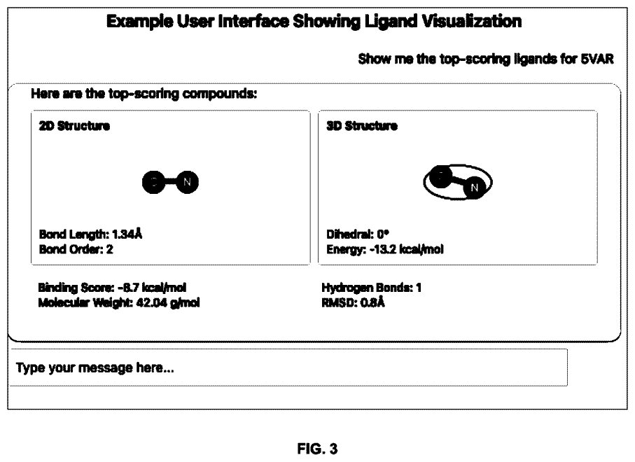

### 5.3 EXAMPLE USER INTERFACE (FIG. 3): EXAMPLE USER INTERFACE SHOWING LIGAND VISUALIZATION

FIG. 3 shows an example user interface 300. The chat message 310 contains text generated by the language model and a ligand visualization 320. The ligand visualization is rendered from an SVG file generated by the backend pipeline or from another artifact reference such as an encoded structure representation.

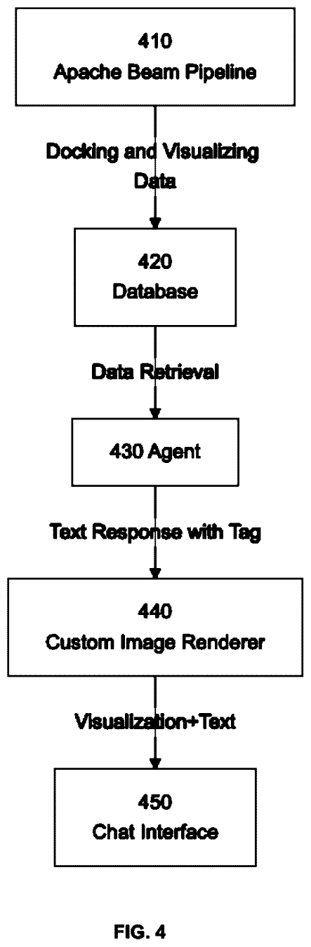

### 5.4 DATA FLOW (FIG. 4): DATA FLOW FROM BACKEND PIPELINE TO FRONTEND DISPLAY

FIG. 4 shows the data flow 400 from the backend pipeline to the frontend display:
410: The Apache Beam pipeline generates the chemical data (ligand-protein complexes, binding energies) and visualizations (for example: SVG files).
420: The data and visualizations are stored in the data storage system. Artifact references are stored and indexed (e.g., paths, URIs, database keys, or opaque handles).
430: The Agent retrieves the data, passes it in an appropriate form to the AI Language Model, and formats it into a text response, including the visualization tag.
440: The frontend resolves the artifact reference (optionally via the resolver service) and retrieves a renderable resource locator (e.g., a signed URL) prior to rendering.
450: The frontend JavaScript code renders the artifact (e.g., SVG or other renderable representation) within the chat interface.
   The system uses a custom image renderer to handle the specific tags.

### 6. POTENTIAL APPLICATIONS

The invention can be applied in any field in which users analyze chemical or molecular data, or in which AI-assisted conversational interfaces are used to process, explain, and present molecular representations and associated computational artifacts. Non-limiting examples include:
Computational Chemistry: Streamlining review of molecular structures, poses, scores, and analyses for research workflows.
Drug Discovery: Facilitating identification, triage, and communication of candidate therapeutic compounds and binding hypotheses.
Pharmaceutical Research: Enhancing analysis and reporting of computational results used in experimental planning and validation.
Synthetic Biology: Assisting in modeling and communicating DNA, RNA, and protein structures and interactions.
Environmental Science: Analyzing pollutants, metabolites, and other molecules and their modeled interactions.
Education and Scientific Communication: Providing interactive molecular models and explanations for learning and presentation.

### 7. ADVANTAGES AND TECHNICAL EFFECTS

The invention provides technical advantages over approaches in which a language model must generate images directly or must re-emit raw visualization data for a client to re-render on each response.
1. Reliable binding of narrative to artifacts: By embedding machine-interpretable visualization directives that reference specific stored artifacts, the system deterministically binds explanatory text to the correct molecule, pose, or complex visualization, reducing ambiguity and preventing display of unintended or hallucinated structures.
2. Reduced compute and bandwidth: Generating images token-by-token (or regenerating visualization data on demand) is computationally expensive and can be slow. By referencing precomputed or cached visualization artifacts via artifact references, the system avoids repeated generation and enables low-latency rendering.
3. Reuse and caching across interactions: Visualization artifacts generated by a pipeline can be reused across multiple conversational turns, users, or downstream reports, enabling caching and eliminating redundant recomputation when a user asks follow-up questions or requests alternative views.
4. Scalability to large result sets: Docking and related pipelines may produce thousands to millions of candidate ligands and associated artifacts. By referencing artifacts through directives and rendering selectively (e.g., top-N ranked results), the system scales to large datasets without requiring a user to manually open separate tools or files.
5. Separation of concerns and model flexibility: The language model focuses on interpretation and explanation while the visualization pipeline and rendering components handle artifact creation and display. This enables use of different language models without retraining image generation capabilities and supports multiple visualization backends and client renderers.
6. Improved safety and correctness controls: In embodiments using opaque handles and a resolver service, the system can validate directive types, enforce authorization, and restrict artifact types that may be retrieved and rendered, improving correctness and reducing risk compared to arbitrary file-path generation or direct model-generated binary outputs.

Compared to approaches in which a language model generates images token-by-token or repeatedly emits raw visualization data for client-side rendering, the disclosed directive-and-artifact-reference approach enables deterministic binding between narrative and specific stored artifacts, supports reuse and caching across conversational turns, reduces compute and bandwidth by avoiding repeated generation, and improves consistency by delegating rendering to purpose-built visualization components operating over validated artifact references.

### APPENDIX A: ARTIFACT MANIFESTS AND VISUALIZATION DIRECTIVES

### A1. Visualization Directive Types

In some embodiments, the conversational agent embeds visualization directives in an otherwise natural-language response. A visualization directive may appear inline in prose, in lists, or inside tabular content. A visualization directive identifies a directive type and an associated artifact reference. Non-limiting examples of directive types include:

### 1. Ligand 2D Depiction From Encoded Structure

![](@ligand_smiles_base64:<base64_smiles>)
where <base64_smiles>comprises a base64-encoded string representing a SMILES string. A client application decodes the value and renders a 2D molecular depiction.

### 2. Ligand or Complex 3D Visualization From Stored Artifact

![](@ligand_visualization:<ligand_uuid>:<context_uuid>)
where <ligand_uuid>identifies a ligand instance (e.g., a pose or structure) and <context_uuid>identifies an associated context such as a target, receptor, run, docking campaign, binding site, or other grouping. A client application resolves the referenced artifact(s) and renders a 3D view (e.g., pose in pocket).

In various embodiments, the directive payload may include additional identifiers, parameters, or metadata, and may be expressed using alternative delimiter schemes, key-value pairs, JSON, XML, or other machine-readable encodings.

Non-limiting list of directives: @metabolism, @pharmacophores, @conformers, @md_trajectory, @rmsd_plot, @rmsf_plot, @interaction_map, @hbonds_map, @grid_map, @difference_map, @pose_clusters, @score_table.

### A2. Resolution and Rendering Rules

In some embodiments, a client application that displays the conversational interface performs one or more of the following steps:
1. Parse a response message and detect visualization directives embedded in the message content, including within table cells.
2. Validate that each directive type is recognized and permitted (e.g., allowlisted).
3. Resolve each artifact reference, including by (i) decoding an encoded structure (e.g., base64 SMILES) or (ii) calling a resolver service to map one or more identifiers (e.g., UUIDs) to one or more renderable visualization assets and/or renderable resource locators (e.g., signed URLs or streaming endpoints).
4. Render the visualization inline within the conversational UI, optionally using different rendering modules depending on directive type (e.g., 2D depiction renderer vs. 3D viewer).
5. Handle errors by displaying a fallback (e.g., an error badge, placeholder image, or link) when resolution or rendering fails.

In some embodiments, resolution is performed by a resolver service that returns a renderable resource locator, such as a signed URL or a streaming endpoint, and rendering metadata, and enforces authorization and artifact-type restrictions, and returns only permitted artifacts for the requesting user or session.

### A3. Example End-To-end Response Containing Tabular Directives

The following is a non-limiting example of a response generated by a conversational agent after completing a docking campaign and performing quality-control analysis (e.g., PoseBusters) for predicted poses:

| Top | Pauling Id | Vendor name | Vendor ID | 2D Visualization |
Docking Score (kcal/mol) | 3D Visualization | Passed Test |
|-----|------------|-------------|-------------|-----------------------------
-|--------------------------|------------------|-------------|
| 1 | 14069 | enamine | Z2588038959 | ![ ]
(@ligand_smiles_base64:Q2MxbmMyYyhbbkhdMSlDQ04oQyg9TyljMWNjYyhOQyg9TyljM2NjY2
NjMy1jM2NjY2NjMyljYzEpYzFjY2NjYzEtMg==) | −13.705 | ![ ]
(@ligand_visualization:cb85c7a5-bb2a-4ab0-a434-9c67206030db:7c4f42e3-41a2-
4329-aad4-fa8c9e1b9e07) | Passed |
| 2 | 14428 | enamine | Z2235801884| ![ ]
(@ligand_smiles_base64:Q2MxY2NjKE5DKD1PKUMyKGMzY2NjNGMoYzMpT0MoRikoRilPNClDQz
IpbmMxLWMxY2NjYyhDKD1PKU8pYzE=) | −12.414 | ![ ](@ligand_visualization:a4d3f7d7-
920f-4a1e-af64-524f1704e5af:7c4f42e3-41a2-4329-aad4-fa8c9e1b9e07) | Passed |
...

The visualization directives shown above include artifact references (e.g., encoded molecular representations and resolver-addressable identifiers) that allow the client application to deterministically retrieve and render the intended 2D and 3D artifacts without requiring the language model to generate images directly or to repeatedly emit full visualization data.

What is claimed is:

1. A system for presenting computational chemistry information within a conversational interface, the system comprising:
a data pipeline configured to generate and store computational chemistry artifacts associated with one or more molecules or molecule-receptor complexes;
one or more processors configured to provide, to a conversational agent comprising an AI language model, information identifying at least a subset of the computational chemistry artifacts; and
a client application configured to display the conversational interface,
wherein the conversational agent is configured to generate a natural-language response comprising one or more visualization directives embedded in the response, each visualization directive including (i) a directive type and (ii) an artifact reference corresponding to a respective computational chemistry artifact, and
wherein the client application is configured to parse the natural-language response, identify the one or more visualization directives, resolve the artifact reference for each visualization directive to obtain a renderable representation of the respective computational chemistry artifact, and render, within the conversational interface, a visualization corresponding to each visualization directive.

2. The system of claim 1, wherein the artifact reference comprises at least one of a filesystem path, a URL, an object-storage URI, a database key, or an opaque handle.

3. The system of claim 1, wherein the client application is configured to validate at least one of (i) the directive type or (ii) the artifact reference prior to rendering the visualization.

4. The system of claim 1, further comprising a resolver service configured to receive the artifact reference and return a renderable resource locator for the respective computational chemistry artifact.

5. The system of claim 4, wherein the resolver service is further configured to return rendering metadata comprising at least one of an artifact type, a format identifier, or a viewer state.

6. The system of claim 4, wherein the resolver service enforces at least one of authorization, tenancy separation, allowlisting of artifact types, or size limits prior to returning the renderable resource locator.

7. The system of claim 1, wherein the one or more visualization directives are embedded within tabular content of the natural-language response, and the client application renders the visualizations within one or more table cells.

8. The system of claim 1, wherein a first visualization directive causes rendering of a 2D molecule depiction and a second visualization directive causes rendering of a 3D visualization of a molecule or molecule-receptor complex.

9. The system of claim 8, wherein the 2D molecule depiction is rendered from an encoded structure representation included in the artifact reference.

10. The system of claim 9, wherein the encoded structure representation comprises a base64-encoded SMILES string.

11. The system of claim 1, wherein the natural-language response comprises a ranked list of candidate ligands and includes a plurality of visualization directives corresponding to a plurality of ranked ligands.

12. The system of claim 1, wherein the data pipeline generates and stores quality-control results associated with predicted poses, and the natural-language response includes a visualization directive that references a quality-control artifact.

13. The system of claim 1, wherein the client application caches at least one renderable representation or renderable resource locator associated with an artifact reference and reuses the cached information in response to a subsequent conversational turn.

14. The system of claim 1, wherein the conversational agent is configured to generate the visualization directives using at least one of:
(i) direct insertion of visualization directives by the AI language model;
(ii) generation of structured intent by the AI language model and insertion of visualization directives by an agent component; or
(iii) generation of visualization directives by tool calls and generation of narrative text by the AI language model.

15. The system of claim 1, wherein the client application, upon failure to resolve an artifact reference or validate a directive type, renders a fallback element within the conversational interface indicating an error or unavailable visualization.

16. A computer-implemented method for presenting computational chemistry information within a conversational interface, the method comprising:
generating and storing, by a data pipeline, computational chemistry artifacts associated with one or more molecules or molecule-receptor complexes;
providing, to a conversational agent comprising an AI language model, information identifying at least a subset of the computational chemistry artifacts;
generating, by the conversational agent, a natural-language response that includes one or more visualization directives embedded in the natural-language response, each visualization directive including (i) a directive type and (ii) an artifact reference corresponding to a respective computational chemistry artifact;
parsing, by a client application displaying the conversational interface, the natural-language response to identify the one or more visualization directives;
resolving, for each visualization directive, the artifact reference to obtain a renderable representation of the respective computational chemistry artifact; and
rendering, by the client application within the conversational interface, a visualization corresponding to each visualization directive.

17. The method of claim 16, wherein resolving the artifact reference comprises sending the artifact reference to a resolver service that returns a signed URL or streaming endpoint for retrieving the respective computational chemistry artifact.

18. The method of claim 16, wherein the one or more visualization directives are embedded within a table, and rendering comprises rendering at least one visualization within a table cell.

19. The method of claim 16, wherein resolving comprises decoding an encoded structure representation included in the artifact reference to generate a 2D depiction.

20. The method of claim 16, further comprising validating at least one of the directive type or artifact reference against an allowlist prior to rendering.

21. A non-transitory computer-readable medium storing instructions that, when executed by one or more processors, cause the one or more processors to perform the method of claim 16.

22. A computer-implemented method performed by one or more servers for presenting computational chemistry artifacts in a conversational interface, comprising: generating and storing computational chemistry artifacts; storing an index that maps artifact references to the computational chemistry artifacts; receiving, from a client application, a request associated with an artifact reference extracted from a natural-language response generated by a conversational agent comprising an AI language model; validating the artifact reference and a corresponding directive type; resolving the artifact reference to obtain a renderable resource locator and rendering metadata; and providing the renderable resource locator and rendering metadata to the client application for inline rendering within the conversational interface.

23. The method of claim 22, wherein validating comprises enforcing authorization and allowlisting of artifact types.

24. The method of claim 22, wherein the renderable resource locator comprises a signed URL or a streaming endpoint and the rendering metadata comprises a viewer state.
